(19) United States
(12) Patent Application Publication
(10) Pub. No.: US 2005/0244299 A1
(43) Pub. Date: Nov. 3, 2005

(54) Automated sequential injection analysis systems for the determination of trace endotoxin levels

(75) Inventors: Purnendu Kumar Dasgupta, Lubbock, TX (US); Ronald Norman Berzofsky, Timonium, MD (US); Matthew Richard Bonen, New Market, MD (US)

Correspondence Address: BANNER & WITCOFF, 1001 G STREET N W, SUITE 1100, WASHINGTON, DC, 20001, US

(73) Assignee: BIOWITTAKER TECHNOLOGIES INC, WALKERSVILLE, MD (US)

(21) Appl. No.: 10/512,888

(22) PCT Filed: Apr. 30, 2003
(86) PCT No.: PCT/US03/13519

§ 371 Date: Jun. 30, 2005

Related U.S. Application Data
(60) Provisional application No. 60/376,268, filed on Apr. 30, 2002.

(51) Int. Cl. G01N 33/00

(52) U.S. Cl. 422/68.1

(57) ABSTRACT

A method used to detect endotoxin uses a system (10) having bidirectional pump (20), multiport valve (30), fluid section valve (170), fluid holding member (50), solenoid valve (60), air source (63) and detector (100). Sample, LAL reagent and chromogenic substrate fluid segments are introduced into the holding member (50) between air segments, the reagents are then mixed and introduced into the detector (100) for measurement The system can be washed with an alkaline solution and followed by cthanol, or washed with tricthylaminc followed by water.

This application claims the benefit of and incorporates by reference co-pending provisional application Ser. No. 60/376,268 filed Apr. 30, 2002.

### FIELD OF THE INVENTION

The invention relates to automated measurement of endotoxin levels.

### BACKGROUND OF THE INVENTION

Bacterial endotoxin is a potentially widespread contaminant of a variety of materials, such as water, food, pharmaceutical products, and parenteral preparations. Bacterial endotoxins (lipopolysaccharides) are released from the outer cell membranes of Gram-negative bacteria during early stages of growth, phagocytic digestion, or autolysis of bacterial cells. Lipopolysaccharides are water-soluble stable molecules that have both hydrophobic and hydrophilic regions. The latter are composed of repeating oligosaccharide side chains attached to a polysaccharide core.

There is considerable variation in the details of the structure of endotoxins derived from different bacteria. While the polysaccharide moiety is responsible for the immunogenic properties of endotoxins, their toxicity is elicited by the hydrophobic part (called ‘lipid A,’ which is virtually invariant in composition across different bacterial species). Even in small doses, the introduction of endotoxins into the circulatory system of either humans or animals is capable of causing a wide spectrum of nonspecific pathophysiological changes, e.g., fever, increased erythrocyte counts, disseminated intravascular coagulation, hypotension, shock, cell death, etc. In large doses, it causes death in most mammals. Early-life exposure to endotoxins exerts long-term effects on endocrine and central nervous system development and increases predisposition to inflammatory diseases. Shanks et al., Proc. Natl. Acad. Sci. 97, 5645-50, 2000; see also Pearson III, in PYROGENS: ENDOTOXINS, LAL TESTING, AND DEPYROGENATION, Pearson III, ed., Marcel Dekker, Inc., NY, 1985, pp. 11-19; URL address http file type, www host server, domain name “bact.wisc.edu,” file name “Bact330/lectureendo/.”

Given current concerns regarding bioterrorism, it is useful to note that inhalation of high concentration of endotoxins causes dry cough and shortness of breath, accompanied by a decrease in lung function and fever. Rylander, in ORGANIC DUSTS: EXPOSURE, EFFECTS AND PREVENTION, Rylander & Jaccobs, eds., Lewis Publishers, Boca Raton, Fla.,1994; Heederik & Douwes, Ann. Agric. Environ. Med. 4, 17-19, 1997. Epidemiological and animal studies show that chronic respiratory exposure to endotoxins may lead to chronic bronchitis and reduced lung function. Rylander, Scand. J Work Environ. Health 11, 199-206, 1985.

It is thus essential to ensure that the endotoxin contents of parenterally administered drugs or other fluids remain below permissible levels (in the US, this is set by the US Food and Drug Administration). Sterile water for injection or irrigation, for example, has a maximum permissible limit of 0.25 Endotoxin Units (EU)/mL (for endotoxin derived from E. coli, 1 EU is approximately 75-200 pg). See the URL address: http file type, www host server, domain name “fda.gov,” file type “ora/inspect_ref/itg/itg40.html”; United States Pharmacopeia, USP 24-NF 19, Suppl. 2, 2761-62; Jul. 1, 2000.

Measurement of Endotoxins

The rabbit pyrogen test (fever induction in a rabbit) was introduced in the U.S. Pharmacopoeia in 1942 for the general testing of pyrogens, which include bacterial endotoxins. The test is slow and qualitative and has largely been replaced by some form of the Limulus amebocyte lysate (LAL) test. In 1964, Levin and Bang discovered that bacterial endotoxins can greatly accelerate the rate of clotting of blood from the horseshoe crab Limulus polyphemus. Levin & Bang, Bull. Johns Hopkins Hosp. 115, 265-74, 1964; see also the URL address: http file type, www host server, domain name “dnr.state.md.us,” file type “fisheries/education/horseshoe/horseshoefacts.html.” By 1987, the US Food and Drug Administration (FDA) published guidelines for the validation of the LAL test as an alternative to the USP Rabbit Pyrogen Test The superiority of the LAL based assay over the rabbit test has been known for some time. See Levin, in ENDOTOXINS AND THEIR DETECTION WITH THE LIMULUS AMEBOCYTE LYSATE TEST, Watson et al., eds., Alan R. Liss, Inc., NY, 1982, 7-24. Berzofsky U.S. Pat. No. 5,310,657 clearly showed that the LAL test is two orders of magnitude more sensitive than the rabbit test and also less expensive, less time consuming, and easier to perform.

LAL contains several protease enzymes responsible for endotoxin induced gel/clot formation. Through a series of cascade reactions, the primary protein component sensitive to endotoxins activates the proclotting enzyme to form the clotting enzyme. Berzofsky & McCullough in IMMUNOLOGY OF INSECTS AND OTHER ARTHROPODS, Gupta, ed., CRC Press, Boca Raton, Fla., 1991, pp. 429-48; Morita et al., Haemostasis 7, 53-64, 1978. The clotting enzyme then transforms coagulogen to coagulin, which self-associates to form a gel.

Presently there are three major versions of LAL tests: the gel-clot assay (Levin & Bang, 1964; Levin, 1982; U.S. Pat. No. 5,310,657), the turbidimetric assay (Levin et al., J Lab. Clin. Med. 75, 903-11, 1970; Cooper et al., J Lab. Clin. Med. 78, 138-48, 1971; Pearson & Weary, J. Lab. Clin. Med. 78, 65-77, 1971); and the colorimetric assay (Teller & Kelly, in BIOMEDICAL APPLICATION OF THE HORSE SHOE CRAB (LIMULDAE), Cohen, ed., Alan R. Liss Inc., NY, 1979, 423-34; Ditter et al., J. Lab. Clin. Med. 78, 65-77, 385-92, 1971; Dubczak et al, Haemostasis 7, 403-14, 1978; Novitsky & Roslansky, in BACRTERIAL ENDOTOXINS: STRUCTURE, BIOMEDICAL SIGNIFICANCE, AND DETECTION WITH THE LIMULUS AMEBOCOYTE LYSATE TEST, Cate et al., eds., Alan R. Liss, Inc., NY, 1985, 181-93; Sturk et al., Haemostasis 7, 117-36, 1978; Iwanaga et al., Haemostasis 7, 183-88, 1978; Tsuji & Martin, Haemostasis 7, 151-66, 1978; Tsuji et al., Appl. Env. Microbiol. 48, 550-55, 1984).

Turbidimetric assays measure turbidity due to gel formation; apparent turbidity is somewhat affected by the size and the number of particles, etc. but this problem can be largely overcome. Ohki et al., FEBS Lett. 120, 217-20, 1980. Turbidity measurement is generally unaffected by color present in the sample. A quartz oscillator has been used to measure the viscosity change that occurs during gelation; this technique allows turbid samples to be analyzed. Novitsky et al., in DETECTION OF BACTERIAL ENDOTOXINS WITH THE LIMULUS AMEBOCYTE LYSATE TEST, Watson et aL, eds., Alan R. Liss, Inc., NY, 1987, pp 189-96.

In a colorimetric assay, a synthetic chromogenic peptide is hydrolyzed by the clotting enzyme to release the terminal colored chromogenic moiety. It provides better quantitation and is less laborious than clotting based methods. It is also more sensitive because the amount of enzyme needed for the hydrolysis of the chromogenic substrate is less than the amount needed to form a clot. Friberger et al., in ENDOTOXINS AND THEIR DETECTION WITH THE LIMULUS AMEBOCYTE LYSATE TEST, pp 195-206.

Turbidimetric and colorimetric assays can be practiced in two modes. In the endpoint mode, turbidity or color is measured after a fixed incubation period. In the kinetic assay mode, which offers greater dynamic range,.the turbidity or color development is measured continuously as a function of time. In the end point assay mode, a colorimetric reaction can be stopped by adding acid or a surfactant. solution (e.g., SDS), and the absorbance can be measured at any time thereafter. In a turbidimetric assay this is not possible; addition of acid also destroys the turbidity.

Automation

A degree of automation of the turbidirmetric end point assay has been achieved with a commercially available system (Muramatsu et al., Anal Chim. Acta 215, 91-98, 1988; Homma et al., Anal Biochem. 204, 398-404, 1992); however, poor correlation with other methods and generally higher results have been observed (Tsuji & Martin, 1978).

For some time now, the chromogenic LAL test is the most widely used. Jorgensen & Alexander, Appl. Environ. Microbiol. 41, 1316-20, 1981; Novitsky et al., Parenteral. Sci. Technol. 36, 11-16, 1982.

A robotic automated system has been developed for the chromogenic test. Tsuji & Martin, 1978. This early system and its subsequent commercial counterparts has impressive capabilities but the overall cost is very high. See Bussey & Tsuji, J. Parenter. Sci. Technol. 38, 228-33, 1984; Martin et al., J. Parenter. Sci. Technol. 40, 61-66, 1986. In fact, the cost is prohibitive for deployment at each point of use, as is necessary, for example, in sterile water testing applications. Rather, most users utilize microplate reader based instrumentation where 96-well plates are manually loaded with samples, standards, and reagents. See the URL address: http file type, www host server, domain name “Cambrex.com,” file name “biosciences/lal/b-EndotoxinDPS-instrument.htm#1.”

It is known in the art to use flow injection analysis or sequential injection analysis when attempting to detect the presence of a species. Conventional sequential injection analysis involves the use of a system comprising, typically, a rotary, multi-position selection valve around which multiple liquid solutions including samples and reagents are arranged. A bi-directional pump is used to draw up volumes of these samples and reagents through respective ports of the selection valve and into a holding coil where the samples and reagents are stacked and then delivered to a detector for analysis. This process causes mixing of the sample and reagent segments leading to chemistry that forms a detectable species before reaching the detector. The detector is typically attached to one port of the rotary valve via which the stacked segments can be made to flow by the pump. Stacking is the process of providing a plurality of aliquots, slugs or segments of fluids in a single conduit, either discrete and apart one slug or aliquot from another or adjacent to one another. Conventional systems can involve the use of a single pump (syringe or peristaltic) and a single rotary selection valve. Conventional multi-position selection valves permit random access of the ports that are connected to the samples, the reagents and the detector. Conventional selection valves that are usable in sequential injection analysis systems are can have between six and twenty-eight ports. Commonly, the section valves have between eight and ten ports. An electronic actuator that, in some instances, moves through the ports in both clockwise and counter-clockwise directions controls the operation of the selection valve. Typically, only one port is accessed at any time. When compared to flow injection analysis, sequential injection analysis systems have the advantage of being able to access an increased number of solutions with just one pump. However, these types of sequential injection analysis systems have not been used to determine the presence of the endotoxins due, at least in part, to the difficulties in cleaning the system between different test samples.

There is, therefore, a need in the art for an affordable, sensitive, and fully automated (“on-line”) endotoxin determination system that can be used for point of use endotoxin determinations.

### SUMMARY OF THE INVENTION

One embodiment of the invention provides a system for detecting the presence of an endotoxin in a fluid. The system comprises a fluid delivery pump for introducing and moving fluids within said system; a fluid selection valve having a plurality of ports, each of said ports adapted to receive at least one fluid therethrough in response to the operation of said fluid delivery pump; a fluid holding member in fluid communication with said fluid selection valve and said fluid delivery pump, wherein selected fluids received by said fluid selection valve are stacked in a predetermined order within said fluid holding member, a solid state detector in fluid communication with said fluid selection valve for receiving the fluids that enter said selection valve from the fluid holding member; a multiport fluid valve in fluid communication with said fluid delivery pump and said fluid holding member; and a solenoid valve for connecting to a source of pressurized air, said solenoid valve being coupled to one of said ports of said multiport valve, said solenoid valve introducing air into said system after the

Turbidimetric assays measure turbidity due to gel formation; apparent turbidity is somewhat affected by the size and the number of particles, etc. but this problem can be largely overcome. Ohki et aL, FEBS Lett. 120, 217-20, 1980. Turbidity measurement is generally unaffected by color present in the sample. A quartz oscillator has been used to measure the viscosity change that occurs during gelation; this technique allows turbid samples to be analyzed. Novitsky et al., in DETECTION OF BACTERIAL ENDOTOXINS WITH THE LIMULUS AMEBOCYTE LYSATE TEST, Watson et al., eds., Alan R. Liss, Inc., NY, 1987, pp 189-96.

In a colorimetric assay, a synthetic chromogenic peptide is hydrolyzed by the clotting enzyme to release the terminal colored chromogenic moiety. It provides better quantitation and is less laborious than clotting based methods. It is also more sensitive because the amount of enzyme needed for the hydrolysis of the chromogenic substrate is less than the amount needed to form a clot. Friberger et aL, in ENDOTOXINS AND THEIR DETECTION WITH THE LIMULUS AMEBOCYTE LYSATE TEST, pp 195-206.

Turbidimetric and colorimetric assays can be practiced in two modes. In the endpoint mode, turbidity or color is measured after a fixed incubation period. In the kinetic assay mode, which offers greater dynamic range,.the turbidity or color development is measured continuously as a function of time. In the end point assay mode, a colorimetric reaction can be stopped by adding acid or a surfactant. solution (e.g., SDS), and the absorbance can be measured at any time thereafter. In a turbidimetric assay this is not possible; addition of acid also destroys the turbidity.

Automation

A degree of automation of the turbidimetric end point assay has been achieved with a commercially available system (Muramatsu et al., Anal Chim. Acta 215, 91-98, 1988; Homma et al., Anal. Biochem. 204, 398-404, 1992); however, poor correlation with other methods and generally higher results have been observed (Tsuji & Martin, 1978).

For some time now, the chromogenic LAL test is the most widely used. Jorgensen & Alexander, Appl. Environ. Microbial 41, 1316-20, 1981; Novitsky et al., Parenteral. Sci. Technol. 36, 11-16, 1982.

A robotic automated system has been developed for the chromogenic test. Tsuji & Martin, 1978. This early system and its subsequent commercial counterparts has impressive capabilities but the overall cost is very high. See Bussey & Tsuji, J. Parenter. Sci. Technol. 38, 228-33, 1984; Martin et al., J. Parenter. Sci. Technol 40, 61-66, 1986. In fact, the cost is prohibitive for deployment at each point of use, as is necessary, for example, in sterile water testing applications. Rather, most users utilize microplate reader based instrumentation where 96-well plates are manually loaded with samples, standards, and reagents. See the URL address: http file type, www host server, domain name “Cambrex.com,” file name “biosciences/lal/b-EndotoxinDPS-instrument.htm#1.”

It is known in the art to use flow injection analysis or sequential injection analysis when attempting to detect the presence of a species. Conventional sequential injection analysis involves the use of a system comprising, typically, a rotary, multi-position selection valve around which multiple liquid solutions including samples and reagents are arranged. A bi-directional pump is used to draw up volumes of these samples and reagents through respective ports of the selection valve and into a holding coil where the samples and reagents are stacked and then delivered to a detector for analysis. This process causes mixing of the sample and reagent segments leading to chemistry that forms a detectable species before reaching the detector. The detector is typically attached to one port of the rotary valve via which the stacked segments can be made to flow by the pump. Stacking is the process of providing a plurality of aliquots, slugs or segments of fluids in a single conduit, either discrete and apart one slug or aliquot from another or adjacent to one another. Conventional systems can involve the use of a single pump (syringe or peristaltic) and a single rotary selection valve. Conventional multi-position selection valves permit random access of the ports that are connected to the samples, the reagents and the detector. Conventional selection valves that are usable in sequential injection analysis systems are can have between six and twenty-eight ports. Commonly, the section valves have between eight and ten ports. An electronic actuator that, in some instances, moves through the ports in both clockwise and counter-clockwise directions controls the operation of the selection valve. Typically, only one port is accessed at any time. When compared to flow injection analysis, sequential injection analysis systems have the advantage of being able to access an increased number of solutions with just one pump. However, these types of sequential injection analysis systems have not been used to determine the presence of the endotoxins due, at least in part, to the difficulties in cleaning the system between different test samples.

There is, therefore, a need in the art for an affordable, sensitive, and fully automated (“on-line”) endotoxin determination system that can be used for point of use endotoxin determinations.

### SUMMARY OF THE INVENTION

One embodiment of the invention provides a system for detecting the presence of an endotoxin in a fluid. The system comprises a fluid delivery pump for introducing and moving fluids within said system; a fluid selection valve having a plurality of ports, each of said ports adapted to receive at least one fluid therethrough in response to the operation of said fluid delivery pump; a fluid holding member in fluid communication with said fluid selection valve and said fluid delivery pump, wherein selected fluids received by said fluid selection valve are stacked in a predetermined order within said fluid holding member, a solid state detector in fluid communication with said fluid selection valve for receiving the fluids that enter said selection valve from the fluid holding member, a multiport fluid valve in fluid communication with said fluid delivery pump and said fluid holding member; and a solenoid valve for connecting to a source of pressurized air, said solenoid valve being coupled to one of said ports of said multiport valve, said soleneid valve introducing air into said system after the fluids from the fluid holding member have been received by said fluid selection valve and said detector.

Another embodiment of the invention provides a system for detecting the presence of an endotoxin in a fluid sample, said system comprising a fluid delivery pump for introducing and moving fluids within said system; a fluid selection valve having a plurality of ports, each of said ports adapted to receive at least one fluid therethrough in response to the operation of said fluid delivery pump; a fluid holding member in fluid communication with said fluid selection valve and said fluid delivery pump, wherein selected fluids received by said fluid selection valve are stacked in a predetermined order within said fluid holding member; a detector in fluid communication with said fluid selection valve for receiving the stacked fluids from the fluid holding member, said detector comprising multiple detector cells each comprising a tube extending within a detector block, a light source, a first fiber optic extending between said light source and said tube, a second fiber optic extending between said tube and a signal photodiode for delivering light from said tube containing the fluid sample, a third fiber optic extending between said light source and a reference photodiode, said third fiber optic for delivering light from the light source to the reference photodiode, and a system for comparing an output of the signal diode with an output from said reference diode to determine if an endotoxin is present within said tube.

Yet another embodiment of the invention provides a method of detecting the presence of an endotoxin in a test fluid sample. The method comprises steps of: (a) introducing gas into a fluid holding member to form a first gas buffer, (b) introducing an LAL reagent fluid, a chromogenic substrate fluid, and a test fluid sample into a fluid holding member to form a stacked fluid slug adjacent to the first gas buffer; (c) introducing gas into the fluid holding member to form a second gas buffer adjacent to the stacked fluid slug; (d) mixing said stacked fluid slug to form a mixed fluid; (e) introducing said mixed fluid into a portion of a detector cell; (f) introducing a first light emitted from a light source into said portion of said detector cell including said mixed fluids; (g) measuring the light emitted from said light source; (h) measuring a second light from within said portion of said detector cell; and (i) comparing the measured first and second lights to determine if an endotoxin has changed the light from the light source introduced into said portion of said detector cell.

Still another embodiment of the invention provides a method of washing an endotoxin detection system, comprising the steps of (a) introducing a basic solution into an endotoxin detection system to remove endotoxin; (b) rinsing the basic solution from the endotoxin detection system with an aqueous solution comprising about 50% ethanol; and (c) rinsing the aqueous solution from the endotoxin detection system with endotoxin-free water.

A further embodiment of the invention provides a method of washing an endotoxin detection system, comprising the steps of (a) rinsing an endotoxin detection system with deionized water, (b) displacing the deionized water with about 0.05% triethylamine (TEA) to remove endotoxin; and (c) displacing the TEA with endotoxin-free water.

Another embodiment of the invention provides a method of maintaining stability of a chromogenic substrate and an LAL reagent in an endotoxin detection system. The method comprises steps of: (a) introducing gas into a fluid holding member to form a first gas buffer; (b) introducing an LAL reagent fluid, a chromogenic substrate fluid, and a test fluid sample into a fluid holding member to form a stacked fluid slug adjacent to the first gas buffer; (c) introducing gas into the fluid holding member to form a second gas buffer adjacent to the stacked fluid slug; and (d) mixing said stacked fluid slug to form a mixed fluid before delivering the sample mixture to a detection portion of the endotoxin detection system.

Even another embodiment of the invention provides a method for testing a fluid to determine whether said fluid contains endotoxin, said fluid being transported through a conduit. The method comprises (1) withdrawing through a first flow path a sample volume of said fluid from said conduit and flowing said sample volume of said fluid into a mixing zone; (2) mixing said sample volume of said fluid in said mixing zone with an amount of LAL reagent and an amount of chromogenic substrate sufficient to detect endotoxin in said sample volume of fluid; (3) flowing said mixture prepared in step (2) from said mixing zone to an endotoxin detector cell through a second flow path which is in flow communication with at least a portion of said first flow path, and (4) determining whether said sample volume of said fluid contains endotoxin.

The invention thus provides automated “on-line” flow analysis systems that can perform a Limulus amebocyte lysate (LAL)-chromogenic substrate kinetic assay for the determination of bacterial endotoxins.

### BRIEF DESCRIPTION OF THE DRAWINGS

FIG. 1 is a schematic diagram of the system for detecting endotoxins according to the present invention.

FIG. 1A illustrates the system of FIG. 1 as part of a water system.

FIG. 2 is a schematic diagram of a detector cell that forms a portion of the system of FIG. 1 according to the present invention.

FIG. 3. Spectroscopic details relevant to the measurement system. Absorption spectra of the chromogenic substrate and pNA and normalized emission spectra of original and interference filter equipped LED.

FIG. 4 Stability of the reagents (combined LAL-substrate stored at 2-4° C., and separate LAL and substrate (stored at room temperature). FIG. 4A, combined. FIG. 4B, separated. The inset shows a typical temporal absorbance profile in the LAL assay at three endotoxin levels.

FIG. 5. Graph showing correlation between the automated system and Kinetic-QCL system.

FIG. 6. Graphs illustrating assay behavior over an 18-day period for three different endotoxin standards. FIG. 6A, 0.05 EU/ml endotoxin standard. FIG. 6B, 0.5 EU/ml endotoxin standard. FIG. 6C, 5.0 EU/ml endotoxin standard.

FIG. 7. Graph showing effects of using fresh lysate.

FIG. 8. Graph showing coagulation reaction times.

FIG. 9. Graph showing results of a wash experiment.

FIG. 10. Graph showing results of a wash experiment using 0.05% triethylamine (TEA).

### DETAILED DESCRIPTION

The invention provides automated endotoxin detection systems (ie., automated “on-line” flow analysis systems) that can perform a Limulus amebocyte lysate (LAL)-chromogenic substrate kinetic assay for the determination of bacterial endotoxins. The systems can be used to test fluid samples from production lines to detect the presence of endotoxin during the preparation of, for example, water, food, drink, pharmaceutical products (including those for animal and human health), and parenteral preparations.

In automated systems of the invention, a test fluid sample is mixed with a chromogenic substrate and an LAL reagent to form an assay mixture at the point of use. Assay mixtures are then delivered to individual detector cells for the simultaneous collection of time-based absorbance data. This automated system determines endotoxin concentration with good accuracy and reproducibility in the range of 0.005-0.5 endotoxin units (EU)/mL (r²≧0.99). Based on three times the standard deviation of a blank and the slope of a calibration curve, systems of the invention can detect endotoxin concentrations of 0.003 EU/mL or lower. The variability of the assay method is less than 5% (n=10). Analysis time required for a 0.005 EU/mL standard typically is less than 100 minutes.

LAL Reagent and Chromogenic Substrate

“LAL reagent” as used herein refers both to amebocyte lysates obtained from horseshoe crabs (e.g., Limulus polyphemus, Carcinoscorpius rotundicauda, Tachypleudus tridentata, or Tachypleudus gigas) and to “synthetic” LAL reagents. Synthetic LAL reagents include, for example, purified horseshoe crab Factor C protein (naturally occurring or recombinant) and, optionally, a surfactant, as described in WO 03/002976. One such reagent, “PyroGene™,” is available from Cambrex Bio Science Walkersville, Inc. LAL reagents preferably are obtained from Cambrex Bio Science Walkersville, Inc. Lyophilized LAL reagent can be reconstituted with 1.4 mL of LAL reagent water (endotoxin-free water) and kept refrigerated until use.

Any chromogenic substrate that can be used to detect an active serine protease (thrombin, trypsin, etc.) (i.e., has the sequence “Arg-chromogenic substrate) can be used in the automated systems disclosed herein. Such substrates are well-known and are commercially available. For example, the buffered chromogenic substrate (p-nitroaniline terminated pentapeptide (Ac-Ile-Glu-Ala-Arg-pNA, S50-640) is suitable and can be reconstituted with LAL reagent water and stored under refrigeration until use. Fluorogenic substrates having the sequence “Arg-fluorogenic substrate” also can be used and are encompassed within the term “chromogenic substrate.”

E. coli 055:B5 lyophilized endotoxin obtained from Cambrex Bio Science Walkersville, Inc. can be used to generate standard curves. Typically, lyophilized endotoxin is reconstituted with endotoxin-free water (LAL reagent water, Cambrex Bio Science Walkersville, Inc.) and vortexed for at least five minutes to yield a concentration of 50 EU/mL. Refrigerated reconstituted endotoxin is stable for at least one month. For the preparation of working standards, the stock solution is warmed to room temperature, vortexed for 5 minutes, diluted with LAL reagent water, and vortexed again before use.

### Embodiments of the Invention

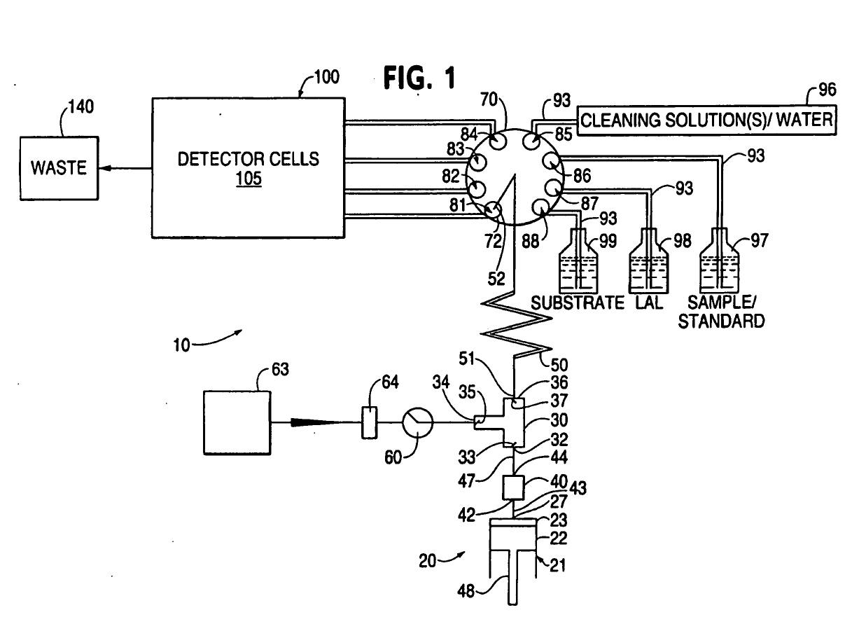

Generally, detection systems of the invention involve withdrawing a sample volume of a fluid from a conduit (e.g., a production line) through a first flow path (as shown in an embodiment in FIG. 1, for example, from vessel 97, 98, or 99 through a selection valve 70 to a holding coil 50), flowing the sample volume into a mixing zone (e.g., the holding coil 50 as shown in FIG. 1), mixing the sample volume of the fluid in the mixing zone with an amount of LAL reagent and an amount of chromogenic substrate sufficient to detect endotoxin in the sample volume of fluid, flowing the mixture of the LAL reagent, the chromogenic substrate, and the sample volume from the mixing zone to an endotoxin detector cell through a second flow path which is in flow communication with at least a portion of said first flow path (as shown in FIG. 1, for example, from the holding coil 50 through the selection valve 70 to the detector 100), and determining whether said sample volume of said fluid contains endotoxin.

FIG. 1 illustrates an automated “on-line” flow analysis system 10 according to the present invention that is configured to substantially perform sequential injection analysis. The system 10 includes a bi-directional fluid delivery pump 20 connected in fluid communication with a three-port valve 30, which can be used as a simple three-port connector. In a first embodiment, the bi-directional fluid delivery pump 20 includes a syringe pump with a syringe 21 having a reciprocating piston 22 positioned within a syringe housing 23 as shown in FIG. 1. A suitable syringe pump that can be used in the system 10 includes 48000 steps and a 500 μL syringe. Such a syringe pump is available from Kloehn Ltd. of Las Vegas, Nev. under the identifier Model No. 50300. Syringe pumps with small volume syringes, such as 500 μl, provide the user with desired levels of precision and accuracy during their operation as part of the sequential injection analysis system 10. As known, a syringe pump is typically driven by a stepper motor that causes the piston 22 within the syringe housing 23 to reciprocate along its path of travel. In one embodiment, the piston 22 of the syringe 21 is moved between each step in response to an electronic control pulse received by the syringe pump. The full “throw” of the syringe 21 (e.g., from a fully reversed position to a fully forward position) corresponds to a known number of pulses and to delivery of the full volume of the syringe 21 in the syringe pump. Therefore, use of a small syringe corresponds to delivery of a smaller volume of fluid per step pulse and, therefore, to more precise flow control. For syringe pumps that utilize gearing systems and motor drives, the flow precision is a function of the ability of the respective drive to stop and start. A computer and software program can be used to control the operation of the fluid delivery pump 20. In a first embodiment, the software program can include WinPump™ software. In an embodiment, it is preferred that any liquids aspirated from their vessel (discussed below) as a result of the operation of the pump 20 do not enter the syringe 21. Easy cleaning of the syringe 21 is facilitated by keeping the syringe clear of the aspirated fluid.

In alternative embodiments, other known bi-directional fluid delivery pumps 20 can be used. For example, in an alternative embodiment the system 10 can include a conventional bi-directional peristaltic pump (not shown). These peristaltic pumps pull fluid from one side of an internal area and push it to an opposing side of the area using a set of rollers and compressible pump tubing.

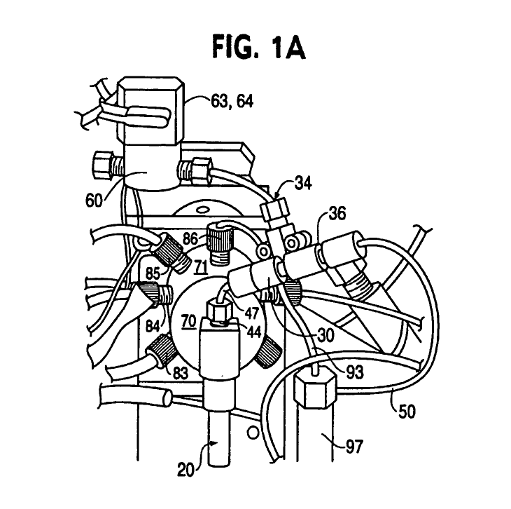

A connection adapter or union 40 can be positioned between the bi-directional fluid delivery pump 20 and the three-port valve 30 for coupling the bi-directional fluid delivery pump 20 to the three-port valve 30. As shown in FIG. 2, the connection adapter 40 can be mounted to the exterior of a selection valve 70 for a simplified arrangement of the components of the system 10. Similarly, the syringe housing 23 can be mounted on the connection adapter 40 as illustrated. In an embodiment, an extended arm 48 replaces a horizontal syringe drive arm that extends from the pump drive motor (not shown) and moves the syringe piston 22 vertically.

The interior of the connection adapter 40 is substantially open so that fluid entering through either an upstream or downstream end of the connection adapter 40 can pass through and exit the connection adapter 40 at its opposite end. The connection adapter 40 includes a first port 42 that is connected to, and in fluid communication with, an outlet/inlet port 27 of the syringe housing 23. As illustrated in FIG. 1, an elongated conduit 43 extends between the inlet/outlet port 27 of the syringe housing 23 and the first port 42. This conduit 43 can include flexible or rigid tubing that has an elongated interior passage through which fluid can flow between the pump 20 and the connection adapter 40. A second port 44 of the connection adapter is connected to and in fluid communication with a first port 32 of the valve 30. As shown in FIG. 1, an elongated conduit 47 extends between the second port 44 and the first port 32. As with conduit 43, conduit 47 can include flexible or rigid tubing that has an elongated interior passage that permits fluid to flow between the connection adapter 40 and the three-port valve 30.

As illustrated in FIG. 1, the three-port valve 30 is substantially T-shaped. The first port 32 of the three-port valve 30 is connected in fluid communication with the connection adapter 40 and the pump 20, as discussed above, so that liquid can be aspirated from one of the peripheral ports of the selection valve 70 via a holding portion 50 of the system 10, such as a holding coil 50 discussed below and via the common port 72 of the selection valve 70. The first port 32 can include a conventional bi-directional valve 33 that permits fluid (including gas) to enter and exit the three-port valve 30. In a first embodiment, the valve 33 includes a two-way flapper valve that permits the ingress and egress of fluids at the first port 32. However, other known bi-directional valves can be used in the present invention.

A second port 34 of the valve 30 is connected to an on/off solenoid valve 60 as shown in FIG. 1. The second port 34 can include a bi-directional valve 35 that permits fluid to enter and exit the three-port valve 30 at the second port 34. Like valve 33, valve 35 can include any known bi-directional valve.

The on/off solenoid valve 60 is in fluid communication with the second port 34 and a filtered compressed air source 63. In an embodiment of the present invention, the solenoid valve 60 includes a conventional solenoid valve. Such a solenoid valve can be obtained from Bio-Chem Valve Corp. of Hanover N.J. under part type 075T2. The solenoid valve 60 can be regulated for a predetermined pressure. In a preferred embodiment, the solenoid valve 60 can be set at 15 psi. The operation of the solenoid valve 60 can be controlled by a computer running WinPump™ software via a programmable digital output and a low current relay. In an alternative embodiment, a 3-way solenoid valve can be in positioned in communication with the pump 20. A filter 64 can be included within the compressed air source 63 or downstream of the air source 63 for filtering any impurities from the air exiting the compressed air source 63. In an embodiment, the filter 64 can include an Acrodisce® glass fiber filter from Pall-Gelman.

A third port 36 of the three-port valve 30 is located downstream of the first two ports 32, 34 as the piston 23 of the syringe pump 20 forces fluid (including air) toward the selection valve 70. The third port 36 receives an end of the holding coil 50 so that the interior of the three-port valve 30 and the holding coil are in fluid communication when the third port 36 is open. A bi-directional valve 37 that permits fluid to enter and exit the three-port valve 30 can be positioned at the third port 36. Like the other bi-directional valves 33, 35, bi-directional valve 37 can include any known valve that allows fluid to enter and exit the three-port valve 30 at the third port 36.

The system 10 also includes the holding coil 50 in which liquid segments (i.e., an LAL reagent, a chromogenic substrate, and a test fluid sample) are “stacked” to form a stacked fluid slug. The holding coil 50 extends between the three-port connector valve 30 and a rotary selection valve 70. As shown in FIG. 1, a first end 51 of the holding coil 50 is connected to, and in fluid communication with, the third port 36 of the three-port connector valve 30. A second end 52 of the holding coil 50 is secured to, and in fluid communication with, a common port 72 of the rotary selection valve 70 as shown in FIG. 1. In an embodiment, the holding coil 50 has a length of about 20 cm and an inner diameter of about 0.86 mm.

The term “coil” is used herein to describe the holding coil 50 because in normal practice a coiled tubing of length, for example, of between about 1 meter and about 5 meters with an internal diameter of about 0.5 mm to about 0.8 mm is used in a tightly coiled or knotted orientation so as to decrease longitudinal dispersion. However, the holding coil 50 is not limited to a coiled length of tubing. Use of the term “coil” is common practice in flow analysis and should not be taken to preclude use of other shaped tubing including straight tubing and knotted tubing, tubing containing beads or other dispersion modifying aids, reactive materials such as particles of solid phase catalysts, or even mixing chambers. In an embodiment, the temperature of the holding coil 50 can be controlled either by means of control computer (not shown) or an auxiliary system (not shown). The contents of the holding coil 50 may also be subjected to external excitation such as ultraviolet light, ultrasound, heat, radiation or microwave energy, with the source of these exciting phenomena being either controlled by control computer (not shown) or by auxiliary systems (not shown).

FIG. 1 also shows a sequential rotary selection valve 70 having a plurality of ports 81-88 through which various fluids and systems can be accessed. As known, these ports 81-88 are in fluid communication with their associated sample containers and/or associated systems. As illustrated, the selection valve 70 can include at least eight ports 81-88. However, the selection valve 70 can include more or less than eight ports. For example, the selection valve 70 can include ten ports. Each port 81-88 is isolated from the other ports 81-88 so that the ports 81-88 are not in fluid communication with each other. One or more of these ports 81-88 can be in fluid communication with a detector 100, as discussed below, where the effluent is driven to waste.

The individual, isolated ports 81-88 of the selection valve are distributed around a common access port 72 that receives the mixed liquids from the holding coil. The common access port 72 can establish a fluid flow path with each of the isolated ports 81-88. The selection valve 70 includes a selector mechanism whereby at least one of the ports 81-88 has access to the common access port 72 so that fluid flow is established between the ports 81-88 and the holding coil 50. As illustrated in FIG. 1A, a housing 71 of the selection valve 70 includes a small bore inert conduit tubing extending from each of the ports 81-88 to form a flow path between the selection valve 70 and either the vessels containing fluids, such as for example, test sample, LAL reagent, chromogenic substrate, wash, and other chemical or biochemical fluids or to detectors 100 or to a waste container or stream or to other sample processing apparatus.

As illustrated in FIG. 1, in a first embodiment of the selection valve 70, four ports 81-84 are each connected to the detector 100 and can operate as exit ports for delivering the mixed liquids to the detector 100. The remaining four depicted ports 85-88 can operate as intake ports and are in fluid communication with members carrying liquids introduced into the system 10 and mixed in the holding coil 50 or employed to clean the holding coil 50. As shown, the first intake port 85 can be connected to a vessel 96 that contains water or a cleaning solution that is capable of removing endotoxins from within the holding coil 50. In this embodiment, the port 85 is in fluid communication with the portion of the vessel or article that contains the cleaning solution via a conduit 93. Intake port 86 can be connected to a vessel 97 for holding a sample liquid to be tested or a solution such that the intake port 86 is in fluid communication with the interior of the vessel 97. As used herein, the term “vessel” includes a container formed of material for holding a test sample or standard, or a portion of a line containing a test sample liquid or the like from a production line or a T port off a flowing fluid line of interest. The second and third intake ports 87, 88 can be connected to, and in fluid communication with, vessels 98, 99 containing LAL and a particular substrate, respectively via conduits 93. Any of the intake ports 85-88 can be connected to any of the above-discussed vessels. The order of connection discussed above for each port 81-88 is for ease of explanation and does not limit the port to a connection with the specifically discussed vessel. The ports 85-88 can be connected to vessels carrying, for example, an LAL reagent solution, a test sample solution, a chromogenic substrate solution, endotoxin standard solutions or a wash solution. As discussed below, the pump 20 and selection valve 70 cooperate to stack within the holding coil 50 zones of reagents and test sample or standard solutions taken from tubes and associated vessels attached to any of the intake valve ports 85-88. As a result, reaction will not ensue until the LAL reagent, chromogenic substrate, and test sample are mixed within the holding coil 50. As discussed, the resulting mixture is then directed to detector 100 via one of the intake ports 81-84.

The detector 100 is schematically illustrated in FIG. 1. FIG. 2 illustrates a detector cell 105 of the detector 100 that includes a detector block 110. The detector block 110 can be formed using any known process. In an embodiment, the detector block 110 is machined from aluminum. The detector block 110 can have any known size. In one embodiment, the exterior dimensions of the detector block 110 are about 30 mm by 20 mm by 20 mm. A tube 120 extends horizontally through openings 124 in the detector block 110. In an embodiment, the tube 120 of each detector cell 105 is formed of a Teflon fluorinated ethylene propylene copolymer (Teflon FEP). Each tube 120 can be 15 gauge and have an inner diameter in the range from 1.0 mm to 3.0 mm, with a preferred inner diameter being about 1.5 mm. The FEP used to form the tubes 120 is more transparent then other commonly used Teflon tubes. Additionally, the thin wall of the tubes 120 promotes better light throughput. FIG. 2 illustrates only a single detector cell 105 for clarity and ease of explanation. However, the system 10 can include a plurality of detector cells 105. The number of detector cells should equal the number of ports on the valve 70 that are dedicated to deliver the mixed liquids to the detector 100. In the illustrated embodiment of FIG. 1, the detector 100 includes at least four detector cells 105. Each detector cell 105 can be operationally isolated form the other detector cells 105 so that contamination between the detector cells 105 does not occur.

Although the following is related to the single detector cell 105 illustrated in FIG. 2, the description is applicable to each of the detector cells 105 of the detector 100. In an embodiment of a detector cell 105 illustrated in FIG. 2, a tube 120 passes through a hole drilled in the detector block 110. Tube 120 forms a tight fit with the respective holes 124 through which it extends. Tube 120 can include a lightweight wall (LW) tube such as those available from Zeus Industrial Products of Orangeburg, S.C. Each tube 120 forms a radial path optical absorbance measurement cell. Each tube 120 includes optical apertures 126 that extend perpendicular to the longitudinal axis 127 of each tube 120. The optical apertures 126 are open on opposite sides of the tube 120 to bring in incident light and carry back transmitted light. The optical apertures 126 can be positioned at any point along the circumference of the tube 120. Tube 120 can have any known size. In one embodiment, tube 120 has a diameter of about 1 mm. A fiber optic 128 can be positioned to extend through the apertures 126 and be securely held within each one of the apertures 126. The fiber optics 128 can be formed of any diameter that will be securely received in the apertures 126. In one embodiment, the fiber optics 128 can include technical grade jacketed acrylic fiber optics with a 1.5 mm core available from Edmund Scientific of Barrington, N.J. In an embodiment, each aperture can include a threaded port so that the fiber optic 128 can be securely held within an aperture 126 by cooperating nuts and ferrules. The aperture 126 is sized smaller than the tube diameter so that light passes through paths that are shorter than the light paths that extend parallel to the diametric paths of the tubes 120. In a radial path detector, light passes not only through the diametric path but also through parallel, shorter paths. The path length can be about 1.15 mm.

The tube 120 is connected to one of the outlet ports 81-84 of the valve 70 via coupled conduit 132 so that the detector cell 105 and the holding coil 50 can be in fluid communication when the common access port 72 and the respective outlet port 81-84 are aligned when open. Conduit 132 can include a length of tubing having an internal passage for delivering mixed liquid from the valve 70 to the tube 120. In one embodiment, the conduit 132 is between about 3 cm and 15 cm in length. The length of the conduit 132 is sized to allow the programmed final displacement of the syringe pump 20 to locate the middle of the final mixed 200 μL slug of the chromogenic substrate, LAL reagent, and test sample in the illuminated region of the detector cell 105. Another length of tubing 123 extends from the detector cell 105 to a waste container 140. As illustrated, the tube 120 extends a length from each side of the detector block 110. The tube 120 can extend between 5 cm and 15 cm on either side of the exterior surface of the block 110. In a preferred embodiment, the tube 120 extends about 10 cm on either side of the block 110. The tube 120 is covered with a material that prevents the intrusion of ambient light into the tube 120. In one embodiment, black heat shrink tubing covers the tube 120 outside the block 110. In another embodiment, metal tubing covers the tube 120 outside the block 110.

The detector cell 105 also includes a light source 150 for the absorbance measurement for each cell, as shown in FIG. 2. The light source 150 includes individual GaN on SiC devices with a nominal center emission wavelength of 430 nm. These light sources 150 are available from Cree Research. The light source 150 of the detector cell 105 can include a LED 154 housed within a threaded opaque polymeric male-male union 160 used for liquid chromatography. In one embodiment, a LED usable in the detector cell 105 can be obtained from LEDtronics of Torrance, Calif. including L200CUB500N-3.8 Vf, with a measured center wavelength of 434-436 nm.

As shown in FIG. 2, legs 155 of the LED 154 extend out through openings 159 of a union 160. The dome area of the LED 154 is removed leaving 1 mm or less of the epoxy polymer on the LED 154. A flat-topped surface 157 of the LED 154 is polished flat. In an embodiment, an interference filter 163 centered at 436 nm of a 10 nm band pass is secured to LED 154 using an optical grade adhesive. A suitable interference filter includes a 4 nm interference filter available from Intor (Soccoro, N.Mex.),

In operation, the LED 154 is driven at about 15 mA (12 V with a 500 series resistor). Fiber 170 carries the light transmitted through the interference filter 163 to the block 110 of the detector cell 105. Fiber 172 carries the light transmitted through the block 110 of the detector cell 105 to the signal photodiode 182. In an embodiment, the signal photodiode 182 is kept in a separate electronics enclosure 183 from the block 110. In one embodiment, a conventional signal photodiode usable in the detector 100 is available from Siemens under part number BPW34. The detector cell 105 also includes a third fiber 176 that collects light from the bottom of the LED 154 and addresses a reference photodiode 186. As illustrated, each electronics enclosure 183 for the detector 100 contains a signal photodiode 182 and a reference photodiode 186. Fibers 170 and 176 are securely connected to the union 160 and hold the LED 154 securely in place. In one embodiment, cooperating nuts and ferrules can form this connection of the fibers 170, 176 to the union 160. The photocurrent from each of the photodiode 182, 186 is converted to voltage using dual JFET operational amplifiers 184, such as those available from Texas Instrument under part number TL082. Each operational amplifier 184 services at least one photodiodes 182, 186 within the detector 100 and each with a nominal gain of 1 V/A. In an embodiment, each operational amplifier 184 can service a pair of photodiodes 182, 186.

According to the present invention, the eight generated voltage signals (two from each detector cell 105) are acquired by signal comparing system 191 that includes a computer 197 through a 12-bit analog-digital converter 193. In one embodiment, the computer 197 includes a Pentium 11 class laptop PC through A/D PC card (PCM-DAS16D 12/AO, Measurement computing of Middleboro, Mass.). Software calculates the ratio of the reference photodiode 186 output to the signal detector photodiode 182 output for each pair of photodiodes 182, 186 and calculates its logarithm to measure absorbance. The results are then compared to detect the presence of an endotoxin. In operation, the four sets of measured absorbance values can be continuously or sequentially displayed on the PC as a scrolling record.

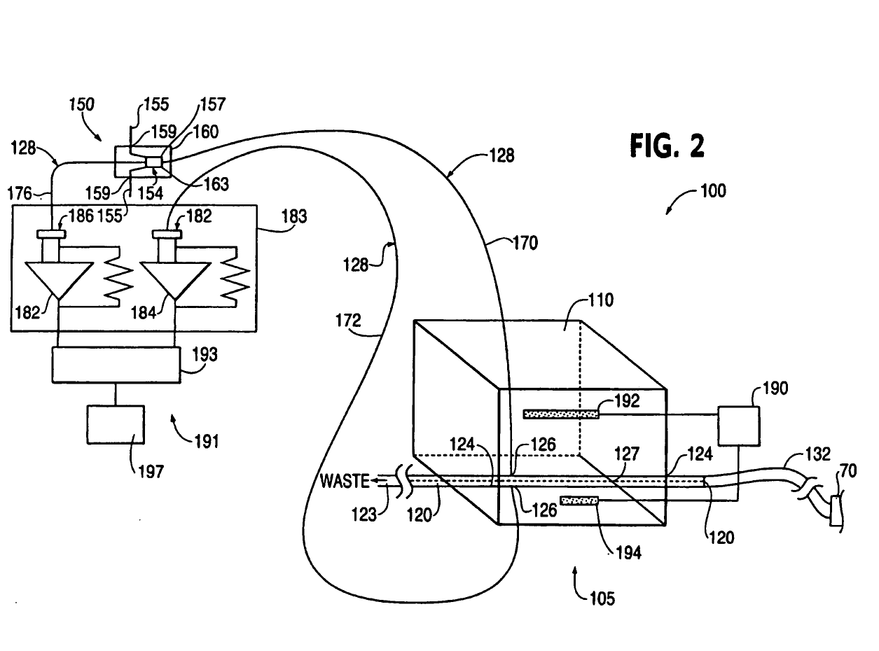

FIG. 3 shows the spectral details relevant to the present system. The pNA generated from the chromogenic substrate absorbs in a broad band centered at 385 nm with a half bandwidth (HBW) of 85 nm. The substrate absorbs with a similarly broad band (HBW 71 nm) but centered further in the UV at 322 nm. There is considerable overlap between the two absorption bands. Because the substrate is obviously present at a much greater concentration, the optimum wavelength to measure the pNA is higher than the wavelength of its absorption maximum to avoid absorption by the substrate. For this reason, commercial instruments relying on this chemistry use a measurement wavelength of 405 nm.

The LEDs 154 or laser diodes at precisely this wavelength are available from, e.g., Cree, Inc., Nichia America Corporation, or Bivar, Inc., and can also be used in the present invention. In an alternative embodiment, any light source that emits in the range of 395-405 nm can be used, thereby possibly increasing photometric sensitivity and allowing the detection of smaller concentrations of pNA.

The normalized emission spectrum of an LED used in an embodiment of the system 10 of the present invention appears in FIG. 3 and exhibits a center wavelength of 434 nm with an HBW of 60 nm. Used as such, the absorbance calibration equation observed for pNA solutions in practice was:
Absorbance=0.0587 [pNA, mM]+0.0152, r²=0.9627

Incorporation of a narrow band (HBW 1 nm) interference filter dramatically improved both sensitivity and linearity.
Absorbance=0.3557 [pNA, mM]+0.0110, r²=0.9995

The kinetics of the LAL reaction can be temperature dependent. The temperature of the detector cell block 110 is maintained constant at 37±0.5° C. by a miniature temperature controller 190 as shown in FIG. 2. A suitable temperature controller is available from Omega Engineering of Stamford, Conn. under part name CN 1632 GNR. The temperature controller 190 can include an imbedded heating element 192. In an embodiment, the heating element 192 includes a cartridge heater with a ⅛ inch diameter available from Watlow of St. Louis, Mo. The temperature controller 190 can also include a point resistance thermometer 194 (RTD). In an embodiment, the point resistance thermometer 194 can include a 100-Ω platinum RTD. The heating element 192 and RTD 194 can be coated with a silicone heat transfer agent to provide good contact with the block 110. Silicone thermal encapsulant can also be positioned about the exterior of the detector block 110 to provide thermal insulation.

In a typical operational sequence, 100 μL of a test sample, 30 μL of an LAL reagent, and 70 μL of a chromogenic substrate solution were sequentially aspirated by the operation of the syringe pump 20. Suitable concentrations of the LAL reagent and chromogenic substrate are taught, for example, in U.S. Pat. No. 5,310,657. A gas, typically air, is introduced through one of the ports of valve 70 before and after aspiration of an LAL reagent, chromogenic substrate, and test sample (which form the stacked fluid slug). As a result, a buffer of gas is positioned at either side of the liquids drawn into the holding coil 50. The contiguous solutions in holding coil 50 were then thoroughly mixed by additional operations of the syringe pump 20 in which the piston 22 reciprocates back and forth within its housing 23. The gas buffers (e.g., air buffers) allow a user to obtain a completely mixed liquid segment such that precise positioning of the segment in the detector 100 does not become an issue that must be considered during the detecting process according to the present invention. Test samples can be withdrawn from a production line; this process can be automated. In an embodiment, multiple stacked fluid slugs comprising aliquots of the same test sample, each separated by a gas buffer, can be positioned within the holding coil 50.

In an embodiment, the piston 22 within the housing 23 is moved at a controlled speed that mixes the liquids within the holding coil 50 without breaking up the liquids or taking an excessive length of time. In an embodiment, the piston 22 makes between two and four complete reciprocating cycles, each cycle including a backward and forward movement, at speeds of between about 4000 and 8000 steps per second. In a preferred embodiment, the speed of the piston is about 6000 steps per second (62.5 μL/s). After the final mixing step of the piston pump 20, the combined liquid is delivered to one of the four detector cells 105 via the holding coil 50 and the selection valve 70.

In another embodiment of the system 10, a valve 70 is designed with at least sixteen ports to accommodate connections to at least six detection cells 105, endotoxin standards of three different concentrations, an LAL reagent, a chromogenic substrate, two cleaning solutions, endotoxin-free water and finally the sample port (configured with a very short line from a recirculating sample loop, as typically used in a water purification system). In this embodiment, the sequence for analysis of six different solutions could include: blank, sample, 0.005 EU/ml standard, sample spiked with a 0.05 EU/mL standard (50 μL+50 μL), 0.05 EU/mL standard, and 0.5 EU/mL standard. In this scenario, no cleaning other than air blow down is needed between samples, and cleaning is performed only after the analysis of the six solutions is over. Commonly available sixteen port selector valves can be used in this embodiment. If a greater number of solutions need to be analyzed, selector valves with as many as twenty-eight ports can be used as discussed above.

Photometric precision of the system can be evaluated by using 0-2 mM buffered pNA solutions that covers an absorbance range of 0-1 AU. Day to day reproducibility is excellent For example, a six-point calibration plot on disparate days yielded the respective linear calibration equations:
Absorbance, AU=0.4929 [pNA, mM]+0.0011, r²=0.9999 (3)
Absorbance, AU=0.4913 [pNA, mM]+0.0004, r²=0.9998 (4)

Stability of LAL Reagent and Chromogenic Substrate

Lysate-substrate reagents for use in chromogenic assays typically consist of a mixture of amebocyte lysate and substrate, which is supplied as a co-lyophilized solid in sterile containers. Immediately before use, the user or a robotic system reconstitutes the lysate-reagent by adding a prescribed amount of endotoxin-free reagent water. Equal amounts of the reconstituted reagent and a test sample are pipetted into microplate wells using standard sterile techniques, and the absorbance is monitored as a function of time. A plot of the logarithm of the time t for the starting absorbance to increase by a fixed amount (typically 0.2 AU) vs. log [endotoxin] is linear with a negative slope (color develops faster as the endotoxin concentration increases). The endotoxin concentration of a sample is determined by reference to a calibration curve generated with endotoxin standards and the same reagent batch, usually on the same microplate.

In systems such as those disclosed herein, the LAL reagent and chromogenic substrate should be reasonably stable. Preferably, these components should not need replacement more often than once a week, otherwise the purpose of automating the system is compromised. The typical combined lysate-substrate reagent is too unstable when kept at room temperature (22-24° C.); even in the absence of any endotoxin, cleavage of pNA from the substrate is rapid. The background absorbance of the blank reagent gets so high within a relatively short period, e.g., 8 hours, that it becomes unusable. In many pharmaceutical plants where sterile water is made, the ambient temperature is often higher than this, and such a reagent preparation and usage protocol will be unsuitable.

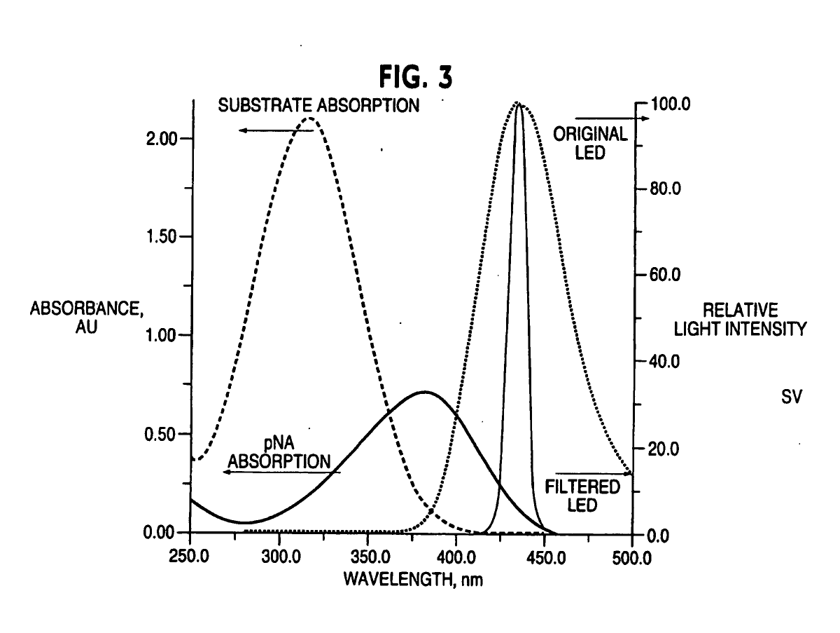

We have found that maintaining the LAL reagent and chromogenic substrate in whatever form (e.g., liquid or lyophilized) separately until their combination at the point of use increases stability of these components. Data demonstrating how calibration changes as a function of the reagent age are summarized in FIG. 4. For the combined lysate-substrate reagent, the biggest change occurs between days 1 and 2, when the reaction time increases. A further small decrease occurs on day 3, but thereafter the reaction time increases monotonously. Between the highest and lowest endotoxin concentrations, the reaction time changes by a maximum of ˜50% at the lowest endotoxin level and by ˜25% at the highest endotoxin level.

In the case of reagents maintained separately until point of use, the reaction time decreases between days 1 and 2 then increases on day 3 past the original day 1 values such that the biggest change occurs between days 2 and 3. Thereafter reaction time increases monotonously. The maximum change in the reaction time during the 8 day period is 31% at the low endotoxin end and 19% at the high endotoxin end. The general direction of change in the reaction time as a function of aging is highly reproducible.

For endotoxin measurement, calibration with standards preferably is tested with the same batch of reagents at about the same time the sample measurement is conducted. Therefore it is not necessary that one make a calibration plot on day 1 and be able to use that plot for a week—with each set of samples, a set of calibration standards will be run. Over a week of use (under the above respective storage conditions), both sets of reagents provide an LOD that is more than adequate for monitoring of regulatory compliance. Separate reagents, if refrigerated, allow operation over a much longer period of time.

pH and Temperature for the Chromogenic LAL Assay

According to the literature, the optimum pH for the activation of the LAL reagent is 7.5, while that for the enzymatic cleavage of pNA from the substrate is 8.2-8.5 (Tsuji et al., Appl. Env. Microbiol. 48, 550-55, 1984; Bussey & Tsuji, J. Parenter. Sci. Technol 38, 228-33, 1984; Dunér, J. Biochem. Biophy. Meth., 26, 131-42, 1993). In a single mixed solution, the optimum pH is 7.7-7.8; the sensitivity is constant in this region (Dunér, 1993). The optimum temperature for the chromogenic LAL assay has been investigated by several researchers and reported to be 37° C. (Bussey & Tsuji 1984; Dunér, 1993). We found that these reported optima apply to the systems disclosed herein as well.

Cleaning Procedures

The present invention also provides cleaning procedures to prevent cross contamination between liquids having the same or different endotoxin concentrations. Contamination is a significant consideration when designing an automated system that is exposed to different concentrations of endotoxin. Even when the system 10 is initially thoroughly cleaned and endotoxin-free, contamination can develop over use due to sample carryover. Contamination is a particular problem because of the propensity of endotoxin to adsorb onto surfaces.

In one embodiment, the system 10 is washed after each set of mixed liquids has been placed into one of the detector cells 105 of the detector 100. In operation, after the mixed liquids pass from the holding coil 50 through the valve 70 and into a detector cell 105, the holding coil 50 and portions of the valve 70 are washed a plurality of times with LAL reagent water for the next sample if the next sample is a repeat of the previous sample. If more than one cell needs to be cleaned, the valve 70 is programmed to rotate to each selected cell and liquid is similarly blown to waste. In one embodiment, the holding coil 50 is washed three times. If a sample or standard with a different, particularly a lower endotoxin concentration is being mixed, then a more stringent cleaning protocol, such as those described below, can be used.

More stringent cleaning protocols can use depyrogenating solutions to remove pyrogens from pharmaceutical processing equipment. One such detergent product, specifically formulated for removing endotoxins (PyroCLEAN™, available from ALerCHEK, Inc.), was indeed effective in removing the endotoxin residuals. However, excessive washing protocols can be required to remove the depyrogenating agent from within the system 10. In another embodiment, strong bases and organic solvents that denature proteins, and aqueous and ethanolic NaOH solutions can be used for the removal of endotoxins. Certain of these bases, solvents and solutions can be used alone and in combination at room temperature and with solutions preheated to temperatures at, and between, about 37° C. and about 60° C., for various residence times and in combination with a nonionic fluorosurfactant, such as Zonyl FSN from DuPont.

The chosen cleaning agent should be in contact with the surface to be cleaned for a predetermined and extended period of time sufficient to remove endotoxin. This time may differ with each cleaning agent. Elevating the temperature of the cleaning agent may increase its effectiveness. In addition, the effectiveness of NaOH solutions in the concentration range of 0.2 to 2 M perceptibly increases with concentration.

In a preferred protocol, a room temperature cleaning protocol is performed. This protocol includes a treatment with about 2 M NaOH being in contact with the interior of the system 10 for a predetermined period of time. The predetermined period of time can be between about five and twenty minutes. In a preferred embodiment, the predetermined period is about ten minutes. In a preferred embodiment, 300 μL of about 2 M NaOH solution is aspirated by the pump 20 through an open port, for example port 85, of the selection valve 70. This NaOH solution is introduced into each detector cell 105 and the holding coil 50. After the NaOH solution has been in contact with the system for ten minutes, the NaOH solution is blown out by the air introduced from the pressurized air source in response to the opening of the solenoid 60. The pressurized air causes the NaOH solution to be removed from the holding coil 50 and each detector cell 105.

Next, the protocol includes repeated treatments of an ethanol:water solution (e.g., about 50% ethanol) and a water wash. In a preferred embodiment, about 300 μL of 1:1 ethanol and water mixture is introduced into each detector cell 105, the valve 70, and the holding coil 50 through a respective port, such as port 85, of the valve 70, and then blown off after about one minute of residence in the system 10. The ethanol:water wash step is then repeated three times, followed by a final wash step with endotoxin-free water. The endotoxin-free water is allowed to reside in the system 10 for about one minute. The system 10 can be cleaned and/or primed prior to use. Priming can utilize the steps and protocols discussed below with respect to the cleaning.

An alternative cleaning protocol is described in Example 3.

In any of these cleaning protocols, the wash liquid can be removed from the system 10 by opening the solenoid valve 60 and introducing compressed air blows into the holding coil 50 and the valve 70. The air blasts drive off the liquid within the holding coil 50, the valve 70 and a detector cell 105 to waste. The pressurized air can be introduced for a predetermined period of time. Such a period of airflow introduction can be between ten seconds and one minute. In a preferred embodiment, the period of airflow introduction can be about 30 seconds.

Performance and Limit of Detection

While there is some difference in the exact reaction times from one reagent batch to another, the typical time required to reach a 20 ΔmAU threshold in systems described above ranges from 7650 s for a blank to 4850 s for 0.005 EU/mL endotoxin. This constitutes a substantial difference and permits good resolution at low concentrations.

All patents, patent applications, and references cited in this disclosure are expressly incorporated herein by reference.

The above discussions do not limit the invention. Although the disclosure describes and illustrates preferred embodiments of the invention, it is to be understood that the invention is not limited to these particular embodiments. Many variations and modifications will now occur to those skilled in the art. For example, the active components of the system, including the pump 20, the three-port valve 30, the selection valve 70 and the detector 100 are controlled by one or more computers via appropriate analog and/or digital communications protocols.

### EXAMPLE 1

Sample Analysis

Different tap water samples, diluted 50 to 100-fold with endotoxin free water, were analyzed at least in duplicate, both by an embodiment of the system described above and by a commercial microplate reader-based instrument. It has been reported that Mg²⁺ accelerates, while Ca²⁺ inhibits, the color development in the chromogenic LAL reaction. See Dewanjee et al., J Nucl. Med. 31, 243-45, 1990. The local tap water contains ˜1.5 mM each of Ca²⁺ and Mg²⁺ and also contains nearly 1300 ppm of total dissolved solids. Recovery of the added endotoxin ranged from 87 to 124%. This would suggest that at least at low levels, presence of common ionic impurities do not cause any significant problems in measuring endotoxins.

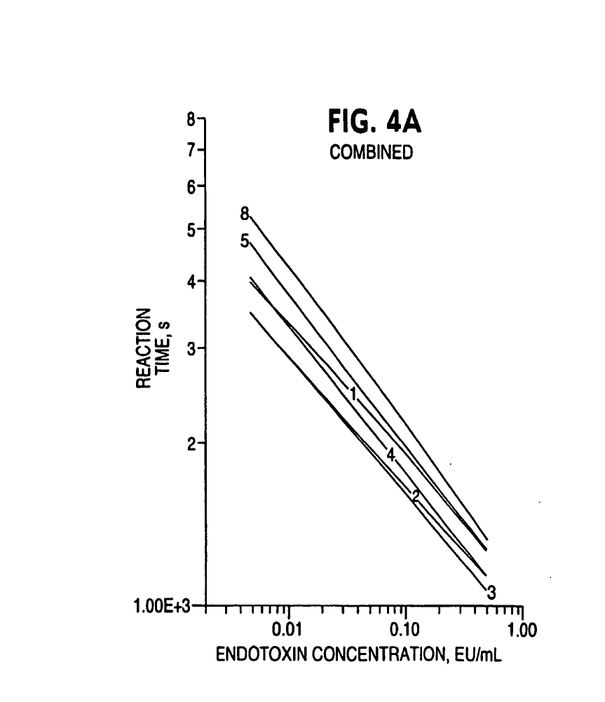

The data generated by the two instruments are plotted in FIG. 5. The best-fit line has a slope (1.0120±0.0568) and intercept (0.0027±0.0097) that are statistically indistinguishable from unity and zero, respectively. At the p=0.05 level, there is no significant difference between the two sets of results.

### EXAMPLE 2

Lysate Properties

The physical properties of the QCL-1000 lysate were studied to provide information on longevity once reconstituted and the ability to store and dispense the lysate. Once reconstituted, the storage temperature of the lysate could be an important variable in determining the useful life of the reagent cartridge. In a temperature controlled lyophilizer, the temperature of the reconstituted lysate was slowly decreased to −4° C. with little change in appearance or viscosity. Depending on the cooling ability of the on-line device, low temperature storage could be used if this would extend the useful life of the lysate.

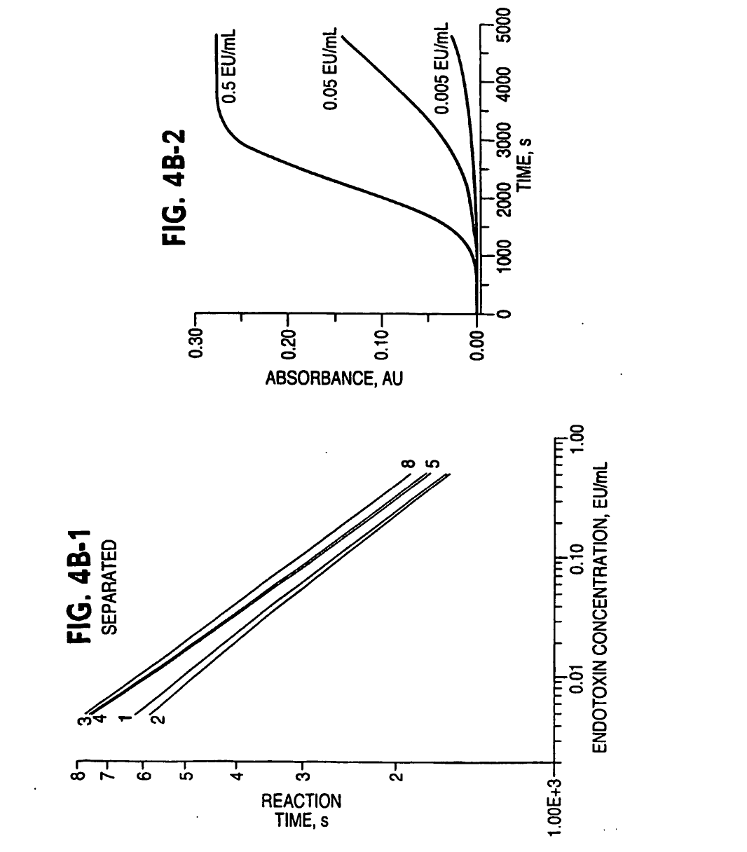

Shelf life studies on the reconstituted reagents were also performed. The endotoxin standards (0.05 EU/ml, 0.5 EU/ml, 5.0 EU/ml), substrate and LAL reagent water were all stored at room temperature and protected from light exposure. Two samples of lysate were used, one stored at 2-8° C. and one stored at 10° C. The latter temperature point was chosen as an achievable set point for a Peltier cooling device incorporated in the on-line system. FIGS. 6A-6C illustrate the assay behavior over an 18 day period.

Based on the data in FIGS. 6A-6C, it was determined that the storage of the lysate at 10° C. improved assay performance, with the reaction times almost always being faster than those using lysate stored at 2-8° C. While reduced temperature could possibly have caused this difference, it is worth noting that the lower temperatures were from storage in a walk-in refrigerator in which temperature variations occur.

Another issue was the length of time that the standard curve could be used. The following tables analyze the running % coefficient of variation (CV) for lysate stored at 10° C.

| Date | Reaction Times (Seconds) | | | | | |
|---|---|---|---|---|---|---|
| Oct. 15, 2001 | 2030 | 2050 | 1190 | 1163 | 748 | 745 |
| Oct. 16, 2001 | 2149 | 2269 | 1457 | 1424 | 889 | 955 |
| Oct. 17, 2001 | 2719 | 2886 | 1614 | 1656 | 976 | 1017 |
| Oct. 18, 2001 | 2894 | 2822 | 1677 | 1705 | 1009 | 1107 |
| Oct. 19, 2001 | 3343 | 2738 | 1855 | 1714 | 1110 | 1115 |
| Oct. 21, 2001 | 3003 | 3254 | 1958 | 1926 | 1222 | 1270 |
| Oct. 22, 2001 | 3110 | 3267 | 1885 | 1889 | 1169 | 1183 |
| Oct. 23, 2001 | 3217 | 3715 | 1993 | 2061 | 1211 | 1323 |
| Oct. 24, 2001 | 3195 | 3528 | 1975 | 2013 | 1202 | 1318 |
| Oct. 29, 2001 | 3752 | 3951 | 2498 | 2429 | 1651 | 1705 |
| Oct. 30, 2001 | 3770 | 3764 | 2368 | 2433 | 1541 | 1609 |
| Oct. 31, 2001 | 4104 | 4523 | 2594 | 2573 | 1702 | 1726 |
| Nov. 1, 2001 | 5643 | 5740 | 3419 | 3479 | 2029 | 2170 |

% CV

| 0.05 EU/ml | 0.5 EU/ml | 5.0 EU/ml |
|---|---|---|
| 5.15 | 11.72 | 12.57 |
| 14.08 | 7.44 | 5.57 |
| 2.86 | 2.31 | 5.46 |
| 9.16 | 4.59 | 4.69 |
| 8.82 | 5.82 | 6.75 |
| 3.98 | 1.80 | 3.74 |
| 8.01 | 4.37 | 5.72 |
| 7.38 | 1.84 | 5.22 |
| 8.99 | 12.25 | 16.81 |
| 2.49 | 2.18 | 4.26 |
| 8.88 | 4.38 | 5.20 |
| 16.29 | 16.59 | 12.07 |

The assay variability never exceeds 25%, even when several days have elapsed between samples. The standard curve can easily be said to be useable during any one 24 hour period.

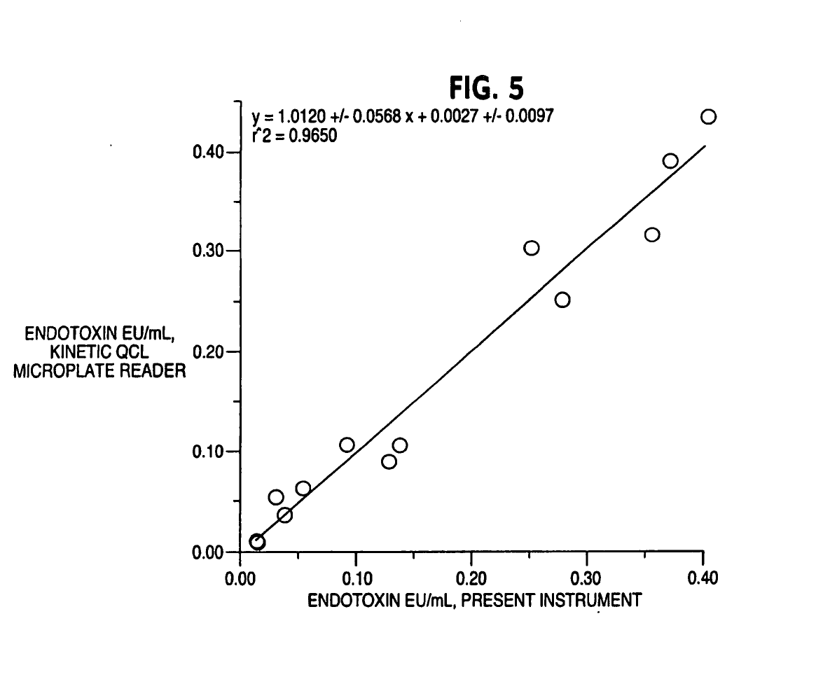

Because all components were aging, an experiment was done to determine if the lysate was the sole source of the increasing reaction times. Using fresh lysate, the data shown in FIG. 7 were obtained. These data indicate that the lysate is the major factor in increasing reaction times.

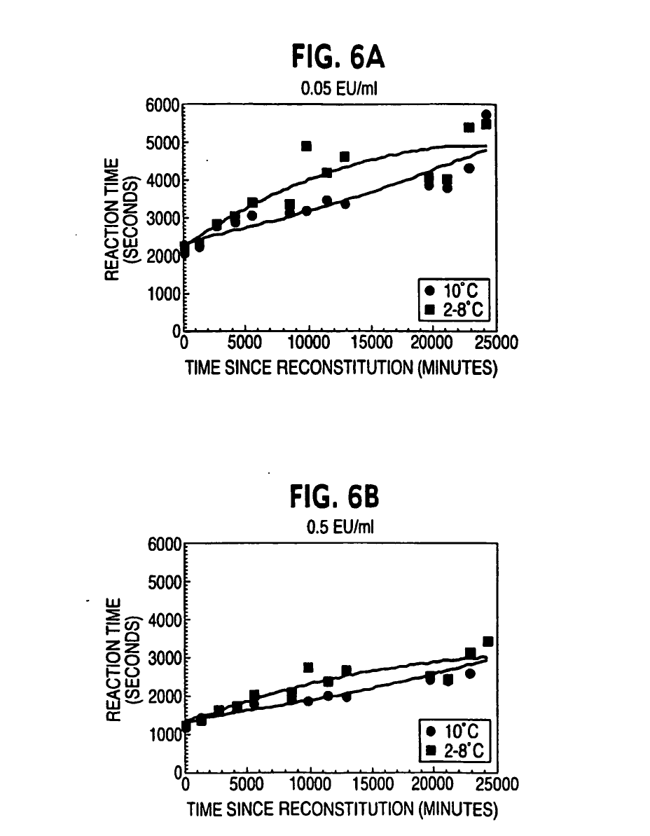

During the aging of the lysate, flocculant material appeared suspended in the vials. This material was present at a higher concentration in the lysate stored at 2-8° C. A product of the coagulation reaction, the flocculant material did not interfere with the overall reaction times (FIG. 8).

Because fluids typically are dispensed and metered in an automated system of the invention using a time and pressure technique, viscosity of the fluids is relevant. During the shelf life experiments, the viscosity of LAL lysate appeared to increase over time. Viscosity measurements taken in a Zeitfuchs cross-arm viscometer showed that the viscosity of LAL lysate increased by 3.5% over the course of the experiments. Density of LAL lysate increased by 3%. (“WFI” is “water for injection,” preferably endotoxin-free water.)

| | | Kinematic | |
|---|---|---|---|
| | Density | Viscosity | Intrinsic Viscosity |
| | (g/ml) | (cSt) | (mPa · s) |
| WFI | 1.0005 | 0.9230 | 0.9235 |
| Fresh Lysate | 0.9893 | 0.9540 | 0.9438 |
| Lysate (18 Days) | 0.9945 | 0.9830 | 0.9776 |

Variations in the volume of fluids introduced into the reaction chamber could also affect the results of the assay. An experiment was performed in which the volume of LAL lysate and chromogenic substrate in the 100 μl of reformed K-QCL reagent was varied and used to detect endotoxin standards. The paired replicates had volume variations where 10% of the reagent was either added or removed. The only replicates that had a % CV greater than 3% were those involving variations in the lysate volume of ±10%. With the 0.5 EU/ml standard, the variation was 3.44%. In the case of the 5 EU/ml standard, the % CV of the paired replicates was 9.44%. Dispensing accuracy need only have a % CV of 5% or less to have a negligible affect on the assay.

### EXAMPLE 3

Comparison of Endotoxin Removal Protocols

Using a quartz cuvette, 600 μl of K-QCL reagent was added to 600 μl of 0.05 EU/ml endotoxin standard. The reaction was then allowed to proceed at 37° C. until Δ0.305 OD had occurred. Referencing a standard curve generated in a microplate experiment, the back prediction indicated an endotoxin concentration in the cuvette of 0.042 EU/ml. A series of blanks were then run to determine if there was a chemical means of removing the endotoxin from the cuvette. One-hour incubations at 37° C. of each of the cleaning solutions was carried out, followed by a rinse with WFI. Solutions of 0.1M NaOH, 0.5M NaOH and 1% deoxycholic acid were tested, and none of the solutions gave satisfactory results. In most cases, the reaction times for the blank solutions did not vary significantly after the cleaning procedure.

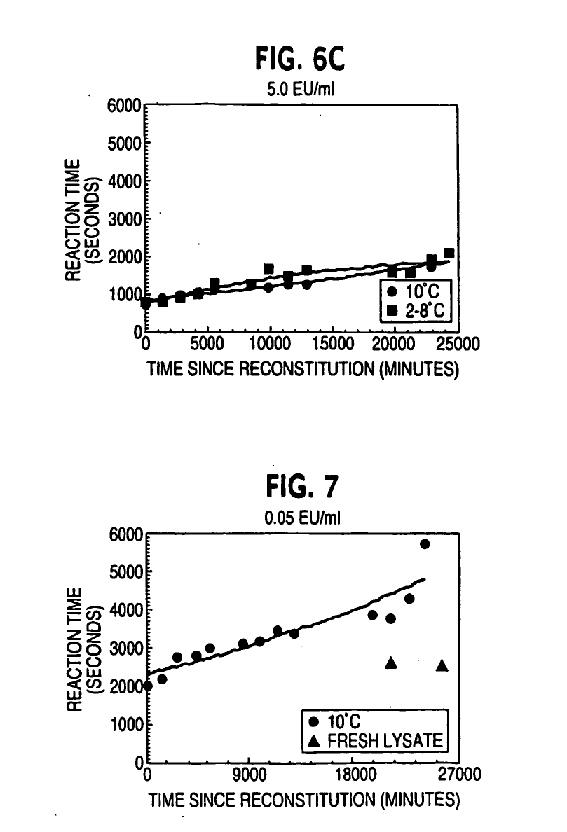

At this point, the cuvette was depyrogenated in a production oven. Following this, a set of standards was run, followed by a wash of the cuvette with WFI. A blank was then run, with a reaction time nearly identical to the last standard. See FIG. 9.

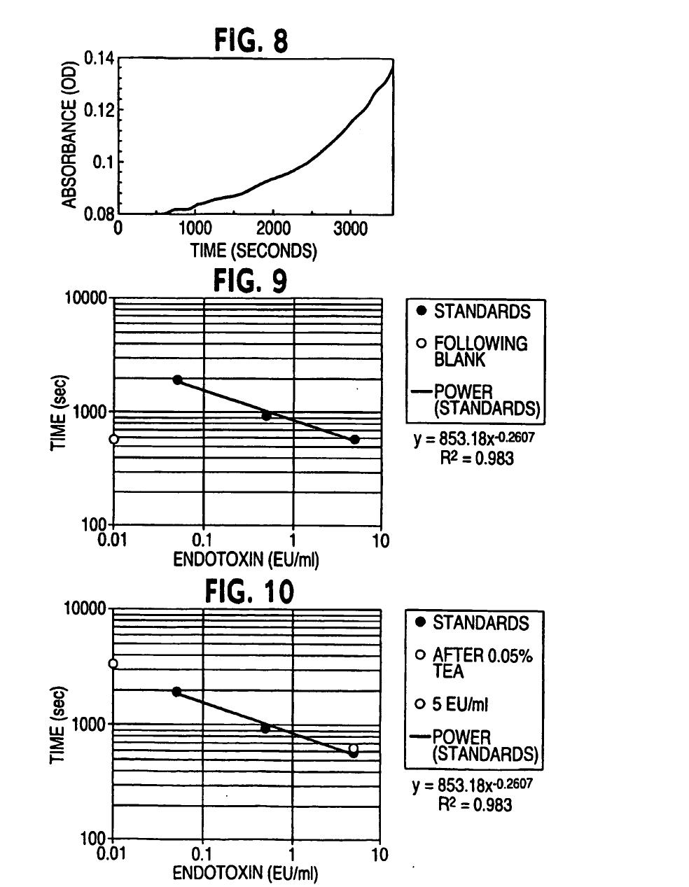

The next cleaning solution that was tried was 0.05% triethylamine (TEA), using the same procedure outlined above. The blank following the washing procedure reacted significantly slower than the lowest standard and was predicted to have a value of 0.01 EU/ml when compared to the standard curve. An assay performed in a microplate to test the inhibitory properties of TEA showed that spike recoveries were down approximately 50% in a solution of 0.05% TEA. While the full-strength solution is mildly inhibitory, thorough washing should reduce it to a level where there is no interference with the LAL assay. See FIG. 10.

The next set of experiments applied the procedure developed in the cuvette to the Pyrex glass reaction chamber. Various standards were incubated in the chamber at 37° C. for 30 minutes, and the solution was then removed from the chamber. A 200 μl sample was read in a microplate at 405 nm.

This experiment generated a reasonable endpoint standard curve. The first blank was measured after running a full set of standards and then cleaning with TEA. A set of standards was then run, followed by the standard cleaning procedure. The following blank was then run. In this case, the endpoint absorbance was lower than that generated by the lowest standard, but not by a significant amount. The blank following the chemical cleaning procedure also produced a higher OD than the first blank run.

An interesting note is shown in the table below. Here, a wash using only DI water was performed after the 5.0 EU/ml standard, followed by a blank. In the reaction chamber, simple rinsing effectively removed a significant percentage of endotoxin, unlike the experiments done in the cuvette. Following this blank the cleaning protocol with TEA was employed, resulting in a further decrease in the background OD produced by the blank test solution.

| | |
|---|---|
| 5 EU/ml | 2.45 |
| Blank | 1.60 |
| Blank | 0.54 |
| 0.05 EU/ml | 0.82 |
| 0.5 EU/ml | 0.98 |
| 5 EU/ml | 1.47 |
| Blank | 0.79 |

1. A system for detecting the presence of an endotoxin in a fluid, said system comprising:
a fluid delivery pump for introducing and moving fluids within said system;
a fluid selection valve having a plurality of ports, each of said ports adapted to receive at least one fluid therethrough in response to the operation of said fluid delivery pump;
a fluid holding member in fluid communication with said fluid selection valve and said fluid delivery pump, wherein selected fluids received by said fluid selection valve are stacked in a predetermined order within said fluid holding member,
a solid state detector in fluid communication with said fluid selection valve for receiving the fluids that enter said selection valve from the fluid holding member;
a multiport fluid valve in fluid communication with said fluid delivery pump and said fluid holding member, and
a solenoid valve for connecting to a source of pressurized air, said solenoid valve being coupled to one of said ports of said multiport valve, said solenoid valve introducing air into said system after the fluids from the fluid holding member have been received by said fluid selection valve and said detector.

2. The system according to claim 1 wherein said solid state detector includes a LED, a reference diode and a signal diode for monitoring a chromogenic reaction within a cell of said detector.

3. The system according to claim 2 wherein said detector further includes a reference photodiode for receiving a light from said LED and a signal photodiode for receiving a light from a tube that contains the fluids received from said holding member, and a system for comparing a signal received from each of the photodiodes.

4. The system according to claim 1 wherein said fluid delivery pump is a bi-directional pump that includes a reciprocating piston.

5. The system according to claim 1 further comprising at least one controller for operating said valves and said pump.

6. The system according to claim 1 further comprising at least one conduit connected with each of said ports of said fluid selection valve and said fluid holding member, whereby selected fluids are selectively transferred between said ports of said fluid selection valve and said fluid holding member.

7. The system according to claim 1 wherein a connector is mounted on an exterior of said selection valve and in a fluid path between said fluid delivery pump and said multiport fluid valve.

8. A system for detecting the presence of an endotoxin in a fluid sample, said system comprising:
a fluid delivery pump for introducing and moving fluids within said system;
a fluid selection valve having a plurality of ports, each of said ports adapted to receive at least one fluid therethrough in response to the operation of said fluid delivery pump;
a fluid holding member in fluid communication with said fluid selection valve and said fluid delivery pump, wherein selected fluids received by said fluid selection valve are stacked in a predetermined order within said fluid holding member;
a detector in fluid communication with said fluid selection valve for receiving the stacked fluids from the fluid holding member, said detector comprising multiple detector cells each comprising a tube extending within a detector block, a light source, a first fiber optic extending between said light source and said tube, a second fiber optic extending between said tube and a signal photodiode for delivering light from said tube containing the fluid sample, a third fiber optic extending between said light source and a reference photodiode, said third fiber optic for delivering light from the light source to the reference photodiode, and a system for comparing an output of the signal diode with an output from said reference diode to determine if an endotoxin is present within said tube.

9. The system according to claim 8 wherein said fluid holding member includes a holding coil in which the selected fluids are stacked.

10. The system according to claim 8 wherein said light source includes a LED.

11. The system according to claim 10 wherein said LED is located within a housing that is isolated from said tube within said detector.

12. The system according to claim 8 wherein said detector further comprises a detector block, and wherein said tube extends through said block.

13. The system according to claim 8 wherein said tube include a Teflon FEP and is in fluid connection with a respective one of the ports of the fluid selection valve.

14. A method of detecting the presence of an endotoxin in a test fluid sample, said method comprising steps of:
(a) introducing gas into a fluid holding member to form a first gas buffer;
(b) introducing an LAL reagent fluid, a chromogenic substrate fluid, and a test fluid sample into a fluid holding member to form a stacked fluid slug adjacent to the first gas buffer,
(c) introducing gas into the fluid holding member to form a second gas buffer adjacent to the stacked fluid slug;
(d) mixing said stacked fluid slug to form a mixed fluid;
(e) introducing said mixed fluid into a portion of a detector cell;
(f) introducing a first light emitted from a light source into said portion of said detector cell including said mixed fluids;
(g) measuring the light emitted from said light source;
(h) measuring a second light from within said portion of said detector cell; and
(i) comparing the measured first and second lights to determine if an endotoxin has changed the light from the light source introduced into said portion of said detector cell.

15. The method according to claim 15 further including repeating at least steps (b)-(e) to form at least a second stacked fluid slug within the fluid holding member.

16. The method according to claim 15 further including the step of washing at least the fluid holding member before repeating steps (b)-(e).

17. The method according to claim 14 further comprising the step of drawing said test fluid sample from a line of fluid.

18. The method according to claim 14 wherein the chromogenic substrate and the LAL reagent are maintained in separate vessels before introduction into the fluid holding member.

19. The method according to claim 14 further comprising the step of forming a plurality of slugs within said fluid holding member.

20. A method of washing an endotoxin detection system, comprising the steps of:
(a) introducing a basic solution into an endotoxin detection detection system to remove endotoxin;
(b) rinsing the basic solution from the endotoxin detection system with an aqueous solution comprising about 50% ethanol; and
(c) rinsing the aqueous solution from the endotoxin detection system with endotoxin-free water.

21. The method of claim 20 further comprising the step of displacing the endotoxin-free water with a gas.

22. The method of claim 20 further comprising the step of displacing fluid in the endotoxin detection system with a gas before the step of introducing the basic solution.

23. The method of claim 20 wherein the basic solution is about 2 M NaOH.

24. The method of claim 20 wherein the predetermined period of time is about 10 minutes.

25. A method of washing an endotoxin detection system, comprising the steps of:
(a) rinsing an endotoxin detection system with deionized water,
(b) displacing the deionized water with about 0.05% triethylamine (TEA) to remove endotoxin; and
(c) displacing the TEA with endotoxin-free water.

26. The method of claim 9 wherein the predetermined period of time is about 1 hour.

27. The method of claim 9 further comprising the step of displacing the endotoxin-free water with a gas.

28. A method of maintaining stability of a chromogenic substrate and an LAL reagent in an endotoxin detection system, comprising the steps of:
(a) introducing gas into a fluid holding member to form a first gas buffer;
(b) introducing an LAL reagent fluid, a chromogenic substrate fluid, and a test fluid sample into a fluid holding member to form a stacked fluid slug adjacent to the first gas buffer;
(c) introducing gas into the fluid holding member to form a second gas buffer adjacent to the stacked fluid slug; and
(d) mixing said stacked fluid slug to form a mixed fluid before delivering the sample mixture to a detection portion of the endotoxin detection system.

29. The method of claim 29 wherein the chromogenic substrate and the LAL reagent are maintained in separate vessels before introduction into the endotoxin detection system.

30. The method of claim 28 wherein the step of mixing is carried out using a pump that includes a reciprocating member.

31. A method for testing a fluid to determine whether said fluid contains endotoxin, said fluid being transported through a conduit, comprising:
(1) withdrawing through a first flow path a sample volume of said fluid from said conduit and flowing said sample volume of said fluid into a mixing zone;
(2) mixing said sample volume of said fluid in said mixing zone with an amount of LAL reagent and an amount of chromogenic substrate sufficient to detect endotoxin in said sample volume of fluid;
(3) flowing said mixture prepared in step (2) from said mixing zone to an endotoxin detector cell through a second flow path which is in flow communication with at least a portion of said first flow path; and
(4) determining whether said sample volume of said fluid contains endotoxin.
